(19) United States
(12) Patent Application Publication
(10) Pub. No.: US 2007/0067031 A1
(43) Pub. Date: Mar. 22, 2007

(54) Intraocular lens

(75) Inventors: Kamal K. Das, Arlington, TX (US); Drew A. Morgan, Crowley, TX (US); Son Trung Tran, Arlington, TX (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/233,210

(22) Filed: Sep. 22, 2005

(51) Int. Cl. A61F 2/16

(52) U.S. Cl. 623/6.17; 623/6.41

(57) ABSTRACT

A an IOL having an edge texture that reduces the light reflected off of the IOL edge and as a result, both the positive and negative dysphotopsia are reduced significantly.

### BACKGROUND OF THE INVENTION

This invention relates generally to the field of intraocular lenses (IOL).

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which an be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

One potential concern with implanted IOLS is photic phenomena, such as, glare, dysphotopsia, stray light, surface reflections, from the IOL that may have detrimental effect on retinal image quality. Some literatures have identified these phenomena as positive and negative dysphotopsia (JA Davison, JCRS, 2000 & Narvaez, et. al., JCRS, 2005). Positive dysphotopsia is related to brightness and streaks of light. According to Davison, “negative dysphotopsia is characterized by a subjective darkness or shadow, which can be arc-shaped, usually in the temporal field.” There is no clear theory that can explain the cause of the negative dysphotopsia type phenomenon. However, there are some clinical observations suggest that light enters the eye at very high angle from the temporal side may create shadow type images on the nasal retina (Trattler et. al., JCRS, 2005).

There have been prior art attempts to reduce or eliminate dysphotopsia. For example, roughening or texturing of the peripheral edge of the IOL and machine specific edge profiles onto the IOL (e.g., U.S. Pat. No. 6,468,306 B1 (Paul, et al.)). None of these prior art methods have been entirely satisfactory. Of course, the lens edge can be roughened sufficiently to produce an opaque edge, but such a surface may limit peripheral vision.

Therefore, a need continues to exist for a safe intraocular lens that substantially reduces dysphotopsia.

### BRIEF SUMMARY OF THE INVENTION

The present invention improves upon the prior art by providing an IOL having an edge texture that reduces the light reflected off of the IOL edge and as a result, both the positive and negative dysphotopsia are reduced significantly.

Accordingly, one objective of the present invention is to provide a safe and biocompatible intraocular lens.

Another objective of the present invention is to provide a safe and biocompatible intraocular lens that is easily implanted in the posterior chamber.

Still another objective of the present invention is to provide a safe and biocompatible intraocular lens that is stable in the posterior chamber.

Still another objective of the present invention is to provide a safe and biocompatible lens that reduces the light reflected off of the IOL edge.

Still another objective of the present invention is to provide a safe and biocompatible lens that reduces both the positive and negative dysphotopsia.

These and other advantages and objectives of the present invention will become apparent from the detailed description and claims that follow.

### BRIEF DESCRIPTION OF THE DRAWINGS

FIG. 1 is a plan view of a typical intraocular lens that may employ the present invention.

FIG. 2 is a graph plotting incident light angle versus light flux.

FIG. 3 is a cross-sectional view of a non-optical outer ring that may be used with the lens illustrated in FIG. 1.

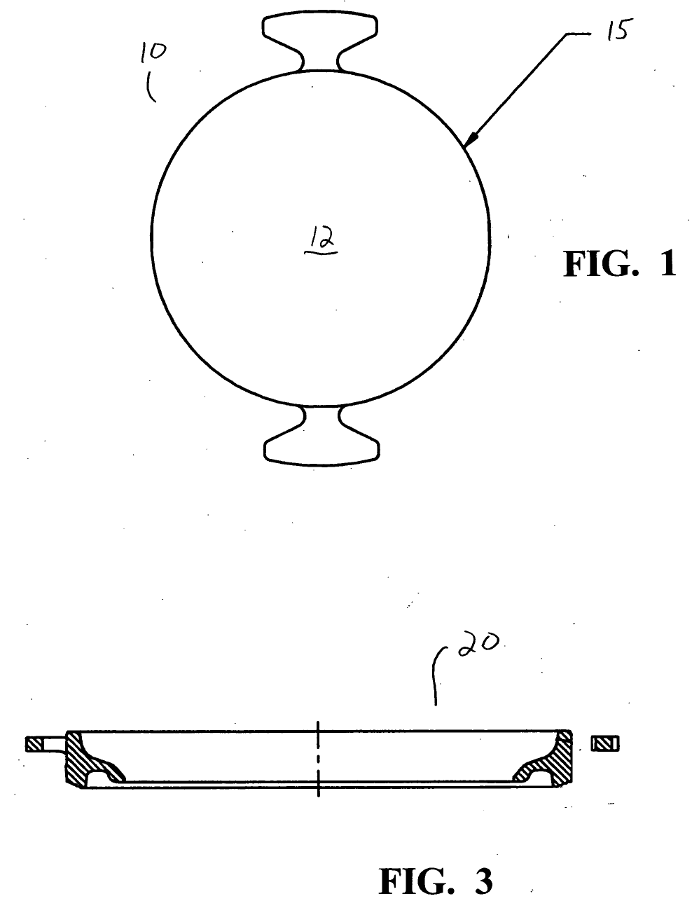

### DETAILED DESCRIPTION OF THE PREFERRED EMBODIMENTS

The present invention uses texture as surface roughness parameter to evaluate the light scattering from the implant surface based on widely known Harvey-Shack scatter model. The most useful and common form of roughness of a surface is root-mean-square (rms) roughness can be described as

$${σ}_{s} = \sqrt{\frac{1}{n}} {\sum }_{x = 0}^{n} {[ s ( x ) - \overline{s ( x )} ]}^{2} . \qquad ( 1 )$$

The rms surface roughness, σₛ, is related to s(x), the surface height at point x in the surface profile and s̅(̅x̅)̅, the average height of the surface profile. The rms surface roughness is the most commonly used parameter for the scattering model and analysis. The rms surface roughness is also related to the scattering properties of the surface. This essentially represents the bidirectional scattering distribution function (BSDF). This BSDF is the ratio of the scattered surface radiance to the incidence surface irradiance.

The total integrated scatter (TIS) from the surface can be computed from the BSDF over the projected solid angle, Ω is the following

$$TIS = {\int }_{Ω} BSDF ⅆ Ω . \qquad ( 2 )$$

This Ω is related to the scattering angle.
Total integrated scatter, TIS, is obtained as
TIS=1−e^{-[2πΔnσ]}. (3)

Where Δn is the refractive index difference across interface.

This model was used to compute the scattered and transmitted components of light from the implants in a model eye using a non-sequential ray tracing program, such as FRED Optical Engineering Software available from Photon Engineering, LLC, Tucson, Ariz.

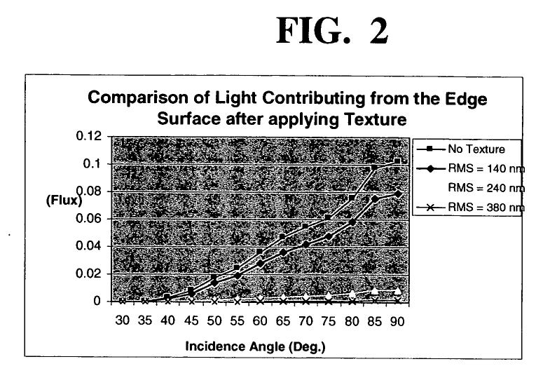

The inventors have discovered that the amount of light going through edge 15 of optic 12 of IOL 10 is not significant for an input angle of 40° or smaller as most of the incidence light is passing through IOL 10 and forming an image on the retina. The inventors have further discovered that at higher angles of incidence, such as, 90°, a significant amount of light (about 10% of the incident light flux) can be reflected, transmitted and scattered from the surface of edge 15 if edge 15 is without any texture on the edge surface. Application of the surface texture as rms surface roughness on edge 15 of optic 12 shows reduction of the edge contribution for all incidence angles. As seen in FIG. 2, the inventors still further discovered that a surface rms roughness (as texture value) of between 340 nm to 380 nm, with approximately 357 nm being most preferred, on the edge surface can reduce significantly the light that may be contributed from edge 15, thereby reducing or eliminating both the positive and negative dysphotopsia.

One skilled in the art will recognize that the invention as described above can be used to modify edge 15 of IOL 10, or to modify other components of a lens system, such as non-optical outer ring 20 shown in FIG. 3, in a two-component lens system, such as described in U.S. Patent Publication No. 20050015145.

This description is given for purposes of illustration and explanation. It will be apparent to those skilled in the relevant art that changes and modifications may be made to the invention described above without departing from its scope or spirit.

We claim:

1. An intraocular lens, comprising:
a) an optic, the optic having an edge; and
b) a surface texturing or roughening on the edge, the texturing or roughening having a texture value of between 340 nm to 380 nm.

2. The lens of claim 1 wherein the texturing or roughening has a texture value of approximately 357 nm.

3. The lens of claim 1 wherein the texture value is determined using the following equation:
$${σ}_{s} = \sqrt{\frac{1}{n}} {\sum }_{x = 0}^{n} {[ s ( x ) - \overline{s ( x )} ]}^{2} .$$

4. The lens of claim 1 wherein the texture value is determined using a non-sequential ray tracing program.

5. An intraocular lens system, comprising:
a) a non-optical outer ring;
b) an optic; and
c) a surface texturing or roughening on the outer ring, the texturing or roughening having a texture value of between 340 nm to 380 nm.

6. The lens system of claim 5 wherein the texturing or roughening has a texture value of approximately 357 nm.

7. The lens system of claim 5 wherein the texture value is determined using the following equation:
$${σ}_{s} = \sqrt{\frac{1}{n}} {\sum }_{x = 0}^{n} {[ s ( x ) - \overline{s ( x )} ]}^{2} .$$

8. The lens system of claim 5 wherein the texture value is determined using a non-sequential ray tracing program.
